(19) United States
(12) Patent Application Publication
(10) Pub. No.: US 2009/0003521 A1
(43) Pub. Date: Jan. 1, 2009

(54) Optimized clinical workflow for combined 2D/3D morphological and functional coronary interventions using a robotic angiography system

(76) Inventors: Estelle Camus, Mountain View, CA (US); Oliver Meissner, Munich (DE); Martin Ostermeier, Buckenhof (DE); Thomas Redel, Poxdorf (DE)

Correspondence Address: BRINKS HOFER GILSON & LIONE, P.O. BOX 10395, CHICAGO, IL, 60610, US

(21) Appl. No.: 11/824,334

(22) Filed: Jun. 28, 2007

(51) Int. Cl. A61B 6/03

(52) U.S. Cl. 378/62

(57) ABSTRACT

A method of optimized diagnosis and treatment of suspected myocardial infarctions is described. The patient having possible coronary artery disease is transferred to a treatment room with an imaging modality suitable for obtaining computed tomographic (CT)-like images. Such images are obtained as radiographic image data, with or without contrast agent, and used in the medical diagnosis. If minimally invasive therapy such as percutaneous transluminal coronal angioplasty (PTCA) is indicated, the patient is prepared for the procedure in the same room, and the procedure performed, where the imaging modality is used to obtain fluoroscopic images to guide the PTCA procedure, or to assess the results of the procedure. The imaging modality may be mounted to a first positioning robot, and a second robot may facilitate the positioning or movement of the patient.

### TECHNICAL FIELD

The present application relates to a method of improving the clinical workflow in diagnosing and treating coronary artery disease, and similar syndromes.

### BACKGROUND

Coronary artery disease (CAD), which is the end result of the accumulation of atheromatous plaques within the walls of the arteries that supply the myocardium, remains the number one cause of disability and death in modern industrialized countries. In 2005 the estimated direct and indirect cost of CAD in the US is $393.5 billion. In the US in 2001 nearly 900.000 Americans experienced a new or recurrent heart attack, or acute myocardial infarction. With the advent of new generation of imaging modalities such as multi-slice scanners, like the Somatom Sensation 64 or the Somatom Definition (available from Siemens AG, Munich, Germany), cardiac computerized tomography (CT) has become a non-invasive alternative for imaging coronary arteries. CT imaging, with or without intravenous iodine-based contrast, can reliably visualize cardiac anatomy and offers the possibility for real time three-dimensional (3D) image reconstruction. However, CT lacks the two-dimensional (2D) angiographic information necessary for direct interventional procedures.

Cardiac magnetic resonance imaging (MRI) is also useful for evaluating the cardiovascular system. In addition to being non-invasive and low risk, MRI may acquire information about the heart as it is beating, and can be used to create moving images of the heart throughout its pumping cycle. This allows MRI to display abnormalities in cardiac chamber contraction and to show abnormal patterns of blood flow in the heart and great vessels. Due to the development of new imaging techniques, MRI has the capability to identify areas of the heart muscle that are not receiving adequate blood supply from the coronary arteries. Aided by the use of a non-iodine-based enhancing agent (for example, Gadolinium-DTPA), MRI can also be used to identify areas of the muscle that have become damaged as a result of infarction (heart attack). One limitation of MRI in the evaluation of CAD has been the inadequate depiction of the coronary arteries, the susceptibility of the images to artifacts associated with metallic objects, and the long examination times. Direct interventional procedures to the coronary region are not currently feasible when using MRI.

Single Photon Emission Computed Tomography (SPECT) may be used for myocardial perfusion imaging to overcome some of the limitations of planar imaging and to improve the localization and quantification of perfusion defects. Cardiac SPECT has been shown to make the detection and localization of myocardial perfusion defects easier both at rest and during stress. The capability of SPECT to localize CAD and assess the extent and severity of perfusion abnormalities is better than using planar imaging. When SPECT is used to image technetium-based myocardial perfusion tracers, global and regional function of the ventricle can be obtained in addition to regional perfusion.

With the use of imaging modalities such as the Siemens Symbia or Biograph, the functional information of nuclear medicine can be combined with the anatomic information and high spatial resolution of CT imaging. However, no direct interventional diagnostic or treatment procedure is possible due to restrictions on access to the patient or to imaging artifacts associated with the treatment apparatus.

Coronary catheterization is a minimally invasive imaging method of choice for interventional therapy of occlusion, stenosis, restenosis, thrombosis or aneurysmal enlargement of coronary arteries. In addition to information on lesion size and luminal narrowing, interventional procedures are able to directly access the region to be treated via the arterial system. In the US, more than 664,000 percutaneous transluminal coronary angioplasties (PTCAs) were performed in 2003. However, coronary angiography offers little or only very limited information about atherosclerotic plaque composition or the functional impact of stenoses/occlusions on the myocardium.

One step to achieve a combination of 2D/3D information with high-resolution will be the introduction of cardiac Dyna-CT (available from Siemens AG, Munich, Germany). Dyna-CT offers the possibility to create cross-sectional images and 3D reconstructions of the heart and the coronary vessels in addition to 2D angiographic imaging. However, with the use of current angiography systems, like the Axiom Artis series from Siemens, to date there exist some limitations. For example, prolonged imaging times limit the exact evaluation of the coronaries, as movement of the heart will lead to artifacts. In addition, the freedom and speed of movement of the current C-arm X-ray concept is limited. Moreover, fast reliable information on myocardial perfusion is not available.

At present, there exist two possible evaluation scenarios for patients suspected of having acute CAD/acute myocardial infarction. In the case of high clinical as well as laboratory (elevated Troponin C levels) suspicion of acute myocardial infarction or acute coronary event, the patient will immediately be sent to the catheterization lab. As such, only limited morphological and no functional imaging will be available prior to intervention as appropriate imaging modalities are not available. In case of clinical suspicion of acute myocardial infarction, but normal Troponin C levels, the patient will either be observed over a time period of at least 4 hours to rule out or confirm Troponin C elevation or have a coronary CT, if available, to rule out or to confirm treatable CAD.

In the high clinical suspicion case, the amount of information is limited, due to lack of volumetric and cross-sectional imagery. Where the patient is merely observed, due to the uncertainty of the diagnosis, or sent to another laboratory for a CT scan, there is a substantial delay in proceeding to treatment, and the emergency room admittance to balloon angioplasty time is increased.

### SUMMARY

An optimized method of diagnosis and treatment of a patient exhibiting symptoms which may be indicative of, for example, coronary artery disease (CAD) is disclosed. The method includes providing a diagnosis and treatment room having an imaging modality, such as a C-arm X-ray device, having the capability of producing CT-like and fluoroscopic images, and the equipment and supplies needed to perform at least one form of minimally invasive treatment. The patient is transported to the treatment room and positioned so that radiographic diagnostic data may be obtained using the imaging modality. The radiographic image data are processed, as is known in the art, so as to obtain computed-tomographic (CT)-like images. Attending medical personnel, either in the treatment room, or using remote access techniques, interpret the CT-like images to determine the nature of the syndrome and to determine if minimally invasive therapy is indicated. If such therapy is to be initiated, the equipment in the treatment room is used to initiate treatment, and the imaging modality may be used to produce fluoroscopic images during the therapy so as to facilitate the positioning of the catheter, the stent or other treatment apparatus.

Depending on the nature of the examination, image data may be obtained with or without the use of contrast media. The imaging data taken prior to the performance of the treatment may be fused with the fluoroscopic data obtained during the treatment so as to assist in visualization of the position of the treatment devices. Further imaging data may be obtained after complement of the treatment.

The method may further include manipulating the position of the imaging modality using a first robot, and manipulating the position of the patient using a second robot.

### BRIEF DESCRIPTION OF THE DRAWING

FIG. 1 is a flow chart showing the optimized method of functional coronary intervention.

### DETAILED DESCRIPTION

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

In the interest of clarity, not all the routine features of the examples herein are described. It will of course be appreciated that in the development of any such actual implementation, numerous implementation-specific decisions must be made to achieve a developers' specific goals, such as compliance with system and business related constraints, and that these goals will vary from one implementation to another.

When describing the medical intervention technique, the terms “non-invasive”, “minimally invasive”, and “invasive” may be used. Generally, the term non-invasive means the administering of a treatment or medication while not introducing any treatment apparatus into the vascular system or opening a bodily cavity. Included in this definition is the administering of substances such as contrast agents using a needle or port into the vascular system. Minimally invasive means the administering of treatment or medication by introducing a device or apparatus through a small aperture in the skin into the vascular or related bodily structures. This includes the treatments known as percutaneous transluminal coronary angioplasty (PCTA), balloon angioplasty, stenting, and the like. Invasive techniques may include conventional surgery such as coronary artery bypass graft surgery (CABG).

A method of clinical workflow is described which may be used in the evaluation and treatment of patients with suspected acute myocardial infarction using a robotic cardio-angiography system. In an aspect, a patient may present in the emergency room with symptoms that may arouse the suspicion of acute myocardial infarction. In such a circumstance, the patient would be rapidly and directly transferred to the catheterization laboratory.

Non-invasive Coronary C-arm CT (CCCT) with intravenous injection of contrast material may be performed to rule out or confirm coronary stenosis occlusion. This may include, for example, a 3D reconstruction of the vascular tree, curved planar reconstructions, and MIP (maximum intensity projection). In a case of exclusion of significant CAD, the patient can immediately be moved from the cardiac emergency unit (CEU), and the appropriate further diagnostics and treatment performed. The CCCT radiographic unit and the associated image processing may produce angiographic and soft tissue computed tomographic (CT) images comparable to, for example, CT equipment, while permitting more convenient access to the patient for ancillary equipment and treatment procedures. Specifically, the CCCT imaging modality does not completely surround the patient as is the situation with axial CT imaging modalities.

The C-arm system may be mounted on a robotic stand, and the patient support structure may also be robotically mounted, so as to increase the flexibility and precision of orienting the patient with respect to the sensors, life support, sensing and treatment equipment. A C-arm X-ray configuration refers to a C-shaped structural member having an X-ray source and an X-ray detector typically mounted at or near the open ends of the “C” such that a central ray of the X-radiation is orthogonal to the surface of a facing X-ray detector. The space within the C-shape of the arm and the aperture to the “C” provides room maneuvering the patient, or for the physician to attend to the patient so as to perform a physical examination or administer treatment with minimal interference from the X-ray device support structure.

In an aspect, the C-arm may be mounted to a robotic device so as to permit rotational movement of the arm about two perpendicular axes in a spherical, circular or elliptical motion. The entire C-arm may also be translated in linear directions to facilitate positioning with respect to the patient.

In another aspect, the X-ray source and the detector may each be mounted to separate robots, so that the motion of the X-ray source and detector may be independently controlled. The motion of the X-ray source and detector may be coordinated by a robotic control so as to maintain an appropriate orientation for obtaining image data. Where the term C-arm X-ray is used, it should be understood to encompass the equivalent use of multiple robots to achieve the same or similar motion control and to obtain image data as described.

When the C-arm X-ray system uses a real-time X-ray detector, the C-arm may be rotated about the patient so that computed-tomography (CT)-like images may be obtained. In such a use, image data acquisition may take approximately 10 seconds with C-arm rotation through approximately 200 degrees. Other data acquisition protocols may be used including gated incremental rotation, or gated X-ray emission, where the gating is synchronized to a phase of a bodily process.

High-resolution and sensitivity digital detector systems for projection radiography are becoming commonplace in the clinical environment, and may be used to facilitate the rapid acquisition of data with the C-arm system. Such digital detectors provide high spatial resolution while having high quantum efficiency. Apart from reducing the patient radiation dosage, such detectors may be highly linear and have sufficient resolution and dynamic range to obtain radiographic image data to be used in CT applications.

Where the diagnosis is treatable CAD, and the examination room is properly equipped, the procedure may be converted, for example, into an intra-arterial PTCA with puncture of the common femoral or brachial artery and placement of a catheter or sheath. Further diagnostic procedures, balloon angioplasty, or stenting may be performed without repositioning the patient. Angioplasty has come to include all manner of vascular interventions typically performed in a minimally-invasive or percutaneous method.

The obtained 3D CT-like images, with and without contrast enhancement, may be used to quantify the extent of narrowing of the blood vessel (stenosis) so as to select an appropriate stent size and type, and to plan optimum angulations for the intervention, which may be transferred to the treatment system. Stent types may include metal stents, drug-eluting stents, and the like. The technology of minimally invasive treatment and the stents and other apparatus used therein is rapidly evolving, and nothing in this description should be interpreted as limiting the type of minimally invasive therapy that may be employed.

Other studies such as pre-interventional 2D perfusion measurement and computational fluid dynamics (CFD) simulation may be performed without transferring the patient to another examination room.

The CCCT imaging modality may be operated by robotically rotating, for example, a C-arm such that an opposed X-ray source and X-ray detector traverse an angular range of at least about 180 degrees about an axis perpendicular to the plane of the C-arm. A CT-like 3D image may be reconstructed from the detected X-ray data. For example, a soft tissue image may be reconstructed using the methods described in US Pg-Pub US 2006/0120507 entitled “Angiographic X-ray Diagnostic Device for Rotational Angiography”, filed on Nov. 21, 2005, which is incorporated herein by reference. The algorithmic and measurement aspects of computed tomography images are being improved, and the processing of the images obtained by the imaging modalities are expected to continue to improve in resolution and dynamic range, speed, of acquisition and rendering of images and in reduction of the patient X-ray dosage.

The term “X-ray” is used to describe any device that uses ionizing radiation to obtain data regarding the opacity of a path through a patient, regardless of the wavelength of the radiation used. The X-ray source may be rotated around the patient along a circular or elliptical path. The X-ray detector may be disposed diametrically opposed to the X-ray source and such that the plane of the detector is perpendicular to the axis of the X-ray source. This orientation may, for example, be maintained by attaching the X-ray source and X-ray detector to a C-arm, a U-arm or the like. This configuration produces a projection-type image.

Image quality may be improved by the use of an electrocardiogram (EKG) or respiration-controlled acquisition or processing of the 2-D projection images used for the synthesis of 3D CT images, or for 4D images (that is, time varying 3D images). One method of using bodily function monitors such as an EKG or respiration monitor is to select the images to be used in the synthesis of a 3D image from portions of the image data set corresponding to similar stages of a heart or respiration cycle. Alternatively, the bodily function monitor may control the movement of the C-arm and the time of obtaining the image data, which may include gating of the X-ray emissions to reduce the patient radiation dose.

The same CCCT, for example, as was used for the diagnosis may be used to fluoroscopically guide the intervention. A 2D fluoroscopic image may be registered with and overlaid on a previously obtained 3D image so as to aid in visualization of the position of the catheter. Alternatively, or additionally magnetic sensing or guidance of the catheter may be performed.

The results of the procedure may be immediately assessed by, for example, additional myocardial perfusion measurement, flow measurements or CFD.

In performing the PCTA, once access into the artery is gained, a sheath introducer is placed in the opening to keep the artery open and control bleeding. Through this sheath, a long, flexible, soft plastic tube called a guiding catheter is pushed. The tip of the guiding catheter is placed at the mouth of the coronary artery. The guiding catheter may also allow for radio-opaque dyes (usually an iodine-based contrast agent) to be injected into the coronary artery, to permit real time X-ray visualization.

A coronary guidewire, which is an extremely thin wire with a radio-opaque flexible tip, is inserted through the guiding catheter and into the coronary artery. While visualizing the scene by real-time (fluoroscopic) X-ray imaging, the cardiologist may guide the wire through the coronary artery to the site of the stenosis or blockage. The tip of the wire may then be passed across the blockage. The cardiologist may control the movement and direction of the guide wire by gently manipulating the end that sits outside the patient.

The guidewire acts as the pathway to the stenosis. The tip of the angioplasty or balloon catheter is hollow and is inserted at the back of the guidewire. The angioplasty catheter is gently pushed forward, until the deflated balloon is inside of the blockage. The balloon is then inflated, and compresses the atheromatous plaque and so as stretches the artery wall.

If an expandable wire mesh tube (stent) was on the balloon, then the stent will be implanted (left behind) to support the new stretched open position of the artery from the inside.

As described, the workflow facilitates the diagnosis and therapy for cases of suspected myocardial infarction. The 2D/3D fluoroscopic, cross-sectional, morphological and functional information is available at the same place and time, and does not require moving the patient between various treatment rooms. Since the patient is not moved between the diagnosis and treatment, the registration of the 2D and the 3D images is facilitated, and this may reduce the patient radiation exposure and the quantity of contrast agent that is needed. Further, when a robotic system is used, the positioning and repositioning the patient is aided and the demands on the clinical staff are reduced. The dedicated workflow procedure may also serve to reduce errors that are attendant to a complex workflow using differing teams of personnel and different equipment to perform the same or similar functions.

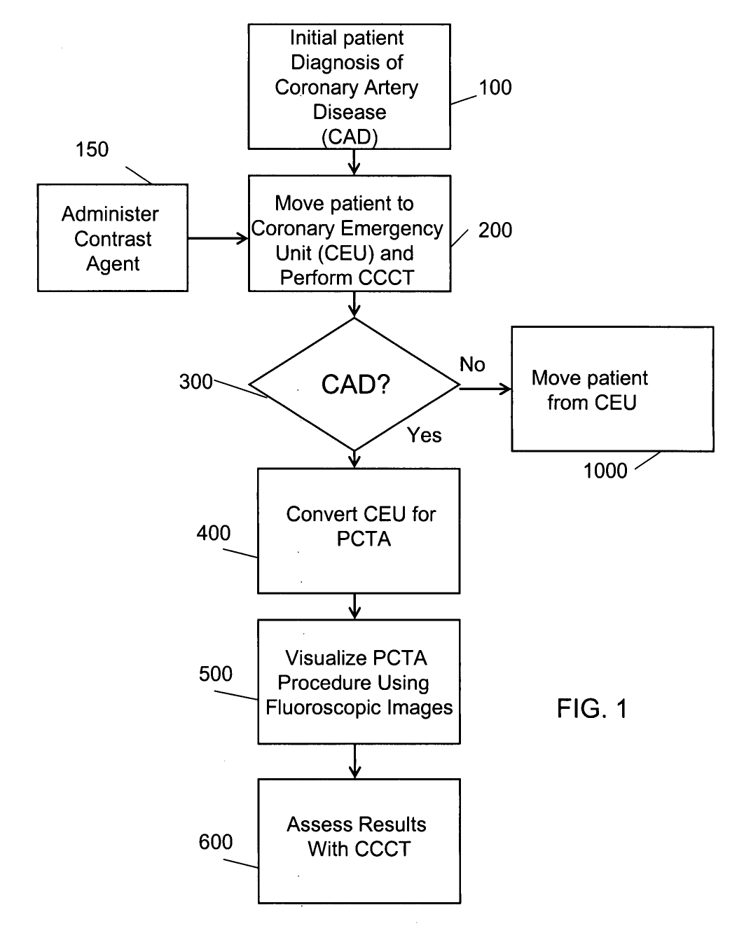

In an aspect, the workflow method may be as shown in FIG. 1. A patient may present in the emergency room, or in a room of the hospital, with symptoms which suggest a possibility of the patient having a myocardial infarction (100). The patient may be moved to the diagnosis and treatment room and prepared for a non-invasive coronary C-arm CT (CCCT) (200). This may include positioning the patient on an examination table and preparing for the administration of contrast material. (150). The CCCT procedure may be performed and the data processed as is known in the art to yield 3D volumetric reconstructions, and 2 D images, as may be needed for diagnostic purposes.

On the basis of the CCCT image data and other diagnostic information, a diagnosis is made, and a course of treatment determined. If there is no coronary artery disease (CAD) (300, NO), or minimally invasive treatment is otherwise contraindicated, the patient is released from the treatment room for alternative therapy or diagnosis (1000). If CAD is diagnosed, and a minimally invasive treatment such as PCTA therapy is indicated, (300, YES), the configuration of the treatment room is made suitable for PCTA, or the like (400). The medical personnel plan the interventional treatment using, for example, the 2D/3D imaging data and select the method of vascular access and, if appropriate, the size, length and type of the stent.

The minimally-invasive PCTA procedures may be performed using the same CCCT or other equipment to visualize the catheter and stent with respect to the patient anatomy (500) in a fluoroscopic mode so as to guide the stent to the appropriate location. After performing the PCTA procedures, the CCCT may be used to perform post-procedure imaging so as to provide an immediate indication of the result of the procedure (600). The patient may then be removed from the treatment room and repositioned for recovery or further treatment. (700). This positioning may be facilitated by robotic equipment.

Where a robotic system is used for the manipulation of the C-arm X-ray, and for the patient support apparatus, a robotic device may be used to transfer the patient from a bed or gurney to the angiographic or surgical table.

One complaint in patients with acute myocardial infarction is dyspnea (shortness of breath), and such patients can be optimally supported in an upright position with a robotic system while enabling complete isocentric C-arm movement.

The methods disclosed herein have been described and shown with reference to particular steps performed in a particular order; however, it will be understood that these steps may be combined, sub-divided, or reordered to from an equivalent method without departing from the teachings of the present invention. Accordingly, unless specifically indicated herein, the order and grouping of steps is not a limitation of the present invention.

Embodiments of this invention may be implemented in hardware, firmware, software, or any combination thereof, and may include instructions stored on a machine-readable medium, which may be read and executed by one or more processors. In an aspect where a computer or a digital circuit is used, signals may be converted from analog format to a digital representation thereof in an analog-to-digital (A/D) converter, as is known in the art. The choice of location of the A/D conversion will depend on the specific system design.

The instructions for implementing processes may be provided on computer-readable storage media. Computer-readable storage media include various types of volatile and nonvolatile storage media. Such storage media may be memories such as a cache, buffer, RAM, flash, removable media, hard drive or other computer readable storage media. The functions, acts or tasks illustrated in the figures or described herein may be performed in response to one or more sets of instructions stored in or on computer readable storage media. The functions, acts or tasks are independent of the particular type of instruction set, storage media, processor or processing strategy and may be performed by software, hardware, integrated circuits, firmware, micro code and the like, operating alone or in combination. The instructions may be stored on a removable media device for reading by local or remote systems. In other embodiments, the instructions may be stored in a remote location for transfer through a computer network, a local or wide area network or over telephone lines. In yet other embodiments, the instructions are stored within a given computer or system.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A method of diagnosis and treatment of a patient, the method comprising:
providing a treatment room having an imaging modality and equipment for performing minimally invasive therapy;
positioning the patient in the treatment room so that radiographic image data are obtained using the imaging modality;
processing the radiographic image data so as to produce computed-tomographic(CT)-like images;
interpreting the CT-like images to determine if minimally invasive therapy is indicated; and
using the imaging modality to produce fluoroscopic images during the therapy if minimally invasive therapy is indicated.

2. The method of claim 1, further comprising administering a radio-opaque contrast agent.

3. The method of claim 1, wherein the minimally invasive therapy is percutaneous transluminal coronal angioplasty (PTCA) treatment.

4. The method of claim 3, where a stent is placed during the PTCA.

5. The method of claim 3, wherein the fluoroscopic images obtained during PCTA treatment are fused with the CT-like images.

6. The method of claim 5, wherein the CT-like images are 2D images.

7. The method of claim 5, wherein the CT-like images are 3D images.

8. The method of claim 1, further comprising mounting the imaging modality to a first robot capable of orienting the imaging modality with respect to a patient.

9. The method of claim 8, wherein the imaging modality is a C-arm X-ray device.

10. The method of claim 9, wherein the patient is positioned with respect to the imaging modality by a second robot.

11. The method of claim 1, further including introducing an intravascular catheter into the patient and guiding the catheter using the fluoroscopic image.

12. The method of claim 1 wherein the radiographic image data is selected to be synchronous with respect to a temporal bodily function.

13. The method of claim 1, wherein the radiographic image data is acquired synchronous with respect to a temporal bodily function

14. The method of claim 13, wherein the bodily function is a cardiac cycle.

15. The method of claim 13, wherein the bodily function is a breathing cycle.

16. The method of claim 1, wherein the patient is positioned on an examination table by a robot.

17. The method of claim 1, wherein the patient is removed from the treatment room without minimally invasive therapy if cardiovascular disease is not diagnosed.
